(19) United States
(12) Patent Application Publication
(10) Pub. No.: US 2007/0148267 A1
(43) Pub. Date: Jun. 28, 2007

(54) Method for producing plant extracts enriched with protease inhibitors for regulation of appetite and food intake in mammals

(76) Inventors: Svyatoslav Komarnytskyy, Highland Park, NJ (US); Ilya Raskin, Manalapan, NJ (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/639,727

(22) Filed: Dec. 15, 2006

Related U.S. Application Data
(60) Provisional application No. 60/750,495, filed on Dec. 15, 2005.

(51) Int. Cl. C12N 9/99; A61K 36/81

(52) U.S. Cl. 424/773; 435/184

(57) ABSTRACT

The present invention relates to a method for manufacturing a potato protein extract enriched with one or more protease inhibitors, such as potato proteinase inhibitor II, potato proteinase inhibitor I, potato cysteine protease inhibitor, and potato carboxypeptidase inhibitor, that offer a significant therapeutic benefit in controlling appetite, food intake, and body weight in mammals. In particular, the present invention relates to a method of obtaining at least one proteinase protein inhibitor from a plant material comprising the steps of extracting plant proteins from the plant material using an aqueous solvent; heating the extract to denature the plant proteins; cooling the extract to precipitate the denatured plant proteins; performing a separation process on the cooled extract to remove the denatured plant proteins; and salting-out the remaining fraction of the plant proteinase inhibitors designated as PPI fraction, and desalting the PPI fraction.

### CROSS REFERENCE TO RELATED APPLICATION

This application claims the benefit of U.S. Provisional Patent Application No. 60/750,495, filed Dec. 15, 2005, the entirety of which is hereby incorporated by reference into this application.

### BACKGROUND OF THE INVENTION

1. Field of the Invention

The present invention relates to preparation of a crude fraction of potato proteinase inhibitors (PPI) that is active in controlling appetite, food intake, and body weight in mammals.

2. Description of Related Art

An economic and simpler method to efficiently recover a fraction of proteinase inhibitors would considerably increase its possibility for therapeutic use in the area of regulation of appetite, food intake, and weight control.

Potato tubers are known as one of the major sources of proteinase inhibitors active in eliciting a satiety response in rodents (U.S. Pat. No. 4,491,578) and humans (Hill et al., Oral administration of proteinase inhibitor II from potatoes reduces energy intake in men, Physiol Bihav 48:241-246 (1990)). However, potato protein recovery is often complicated by interactions with non-protein components of potato tubers. Potato protein recovery in industrial settings is presently achieved through heat coagulation by steam injection after pH adjustment (van Kaningsveld G. A., Physico-chemical and functional properties of potato proteins, Ph.D. Thesis, University of Wageningen, Netherlands (2001)). This method is efficient in removing the coagulated protein, but it also leads to poor solubility and reduced biological activity of the protein fraction, which hampers the potential pharmaceutical applications.

Several other methods for extraction and purification of potato proteinase inhibitors have been reported on a smaller scale.

In 1972, Melville and Ryan reported isolation and purification of chymotrypsin inhibitor I with molecular weight of 39 kDa, as described in Melville et al., Chymotrypsin inhibitor I from potatoes, J Biol Chem 247:3445-3453 (1972). The potato tubers were sliced, soaked in a sodium dithionite solution, and homogenized. The resulting potato slurry was partially clarified by filtering through a nylon mesh, adjusting to pH 3 and centrifuging at 1000 g for 15 min. The supernatant collected after this step was fractionated by ammonium sulfate. Further purification was achieved by heat treatment at 80° C. for 10 min. The clear filtrate of the heated fractions was lyophilized and dialyzed against water for 48 hours. Resuspended extract was then applied to Sephadex G-75 column, and fractions containing the chymotrypsin inhibitor I were pooled, evaporated, and desalted on Sephadex G-25 column, resulting in approximately 90% pure chymotrypsin inhibitor I protein.

In 1976, Bryant et al. reported the isolation of proteinase inhibitor II (molecular weight of 21 kDa) from potato tubers, see Bryant et al., Proteinase inhibitor II from potatoes: isolation and characterization of its protomer components, Biochemistry 15:3418-3424 (1976). The proteinase inhibitor II protein was extracted closely following the procedure used for the isolation of chymotrypsin inhibitor I. In both cases, these methods were expensive, time consuming, and produced low yields of potato proteinase inhibitors.

In 2001, van Kaningsveld described three groups of potato proteins present in potato tuber juice, including the fraction of protease inhibitors that makes up about 50% of the total soluble protein in the potato tuber (van Kaningsveld, 2001). Addition of various strong and weak acids caused precipitation of about 60% of total protein at pH 3. It was also established that the majority of potato proteins unfold between 55° C. and 75° C., and that the increase in the ionic strength from 15 to 200 mM generally caused a significant increase in the denaturation temperature. The presence of ethanol in the extraction solution significantly reduced the denaturation temperature of potato proteins, as well as decreased the pasty consistency of the potato extract.

The isolation of proteinase inhibitor proteins from potatoes is also described in U.S. Pat. No. 6,414,124 (the '124 patent). Proteins from potato tubers are extracted in an aqueous/alcohol extraction medium such as dilute formic acid and 20% ethanol. The alcohol extract is heated to a first temperature of 50° C. or higher and then cooled to a second temperature of 27° C. or lower to form an insoluble precipitate phase containing debris and a soluble phase that contains the heat stable proteinase inhibitor proteins. The heat stable proteinase inhibitor proteins are precipitated from the soluble phase by dialysis against a suitable dialysis medium, such as 0.88% formic acid.

The isolation of the proteinase inhibitor II using ultrafiltration is described in WO 03/003837 A1. The extraction protocol follows closely that of the '124 patent. A molecular weight cut-off for 10 kDa was determined to minimize the loss of the proteinase inhibitor II through the membrane, while removing a large percentage of the impurities. The flow rate was optimized to 0.4 liters per minute per square foot of membrane surface, with a 20 psi pressure differential. Ammonium bicarbonate was selected as an ideal diafiltration buffer.

A method for enhancing the extraction of the proteinase inhibitor II is described in WO 03/003838 A1. A method for controlling the yield and purity of proteinase inhibitor II during extraction is also described in WO 03/003835 A1 and a method for raw material selection and analysis for the isolation of protease inhibitor II from whole potatoes is described in US 2003/0113829 A1. All of these methods use an extraction solution containing sodium chloride and formic acid, similar to the '124 patent.

Pouvreau et al. described relative abundance and inhibitory distribution of protease inhibitors in potato juice. (Pouvreau L., Gruppen H., Piersma S. R., van den Broek L. A. M., van Kaningsveld G. A., Voragen A. G J., Relative abundance and inhibitory distribution of protease inhibitors in potato juice from cv Elkana, J Agric Food Chem 49:2864-2874 (2001)). The proteinase inhibitors were purified using chromatography and represented approximately 50% of the total soluble proteins in the potato juice, with potato proteinase inhibitor II family accounting for 22% of all proteins.

Water-soluble potato proteins were also extracted by a two step ion-exchange chromatographic procedure (Ralet M C, Gueguen J Fractionation of potato proteins: solubility, thermal coagulation and emulsifying properties, Lebensm Wiss Technol 33:380-387 (2000)). Tap water and sodium metabisulfite were added to the potato slices and the mixture was homogenized. The slurry was centrifuged at 2000 g for 15 min. The supernatant was then extensively dialyzed against deionized water and freeze-dried to give potato raw protein fraction. Anion-exchange chromatography was performed on DEAE-Sepharose Fast Flow, and the unbound fraction was further chromatographed on SP-Sepharose Fast Flow. The bound material was eluted as a “basic fraction” containing proteinase inhibitors.

Fritz et al. described the usefulness of immobilized trypsin or chymotrypsin for purification of proteinase inhibitors. See Fritz H., Schult H., Hutzel M., Wiedenmann M., Werle E. On protease inhibitors. IV. Isolation of protease inhibitors with the aid of water insoluble enzyme resins, Hoppe Seylers Z Physiol Chem 348:308-312 (1967).

Recently potato proteinase inhibitors have been implicated in playing a role in extending satiety response, reducing the risk of skin cancer by blocking the UV-induced skin damage, delaying the rate of gastric emptying and reducing glucose and insulin levels after a meal in type II diabetes patients. U.S. patent application Ser. No. 09/624,922 describes a nutritional drink containing potato proteinase inhibitor II. Upon consumption, a significant reduction of hunger was reported for up to 3 and half hours after treatment. Potato proteinase inhibitor II was also shown to delay the rate of gastric emptying in recently diagnosed type II diabetic patients and to improve glucose and insulin levels after a liquid meal (Schwartz J G, Guan D, Green G M, Phillips W T Treatment with an oral proteinase inhibitor slows gastric emptying and actually reduces glucose and insulin levels after a liquid meal in type II diabetic patients, Diabetes Care 17: 255-262 (1994)).

A large number of proteinase inhibitors have been identified from potatoes and related Solanaceae plants. Proteinase inhibitors account for up to 25% of the soluble protein of the potato tuber; including a variety of inhibitors of serine endopeptidases, metallo-carboxy-peptidases, and cysteine proteases. Potato proteinase inhibitors are constantly produced in the tubers, while they accumulate in potato foliage only as a direct result of wounding, either mechanical or following attack by insects (Birk Y., Plant protease inhibitors, Springer Publishing: New York. p. 170 (2002).

Therefore, an immediate need exists for an improved large-scale industrial process for production of potato proteinase inhibitors in a cost-effective way.

### SUMMARY OF THE INVENTION

The present invention relates to a method for manufacturing a potato protein extract enriched with one or more protease inhibitors, such as potato proteinase inhibitor II, potato proteinase inhibitor I, potato cysteine protease inhibitor, and potato carboxypeptidase inhibitor, that offer a significant therapeutic benefit in controlling appetite, food intake, and body weight in mammals. Protein proteinase inhibitors of plant origin include hundreds of inhibitors dispersed among different botanical families. Solanaceae species, as well as legumes and grains, are the richest sources of plant proteinase inhibitors. Several different kinds of inhibitors can be present in a single tissue as it was shown for soybeans, barley grains and potato tubers. The multiplicity of plant proteinase inhibitors may be partly ascribed to the mixed-association of several monomeric subunits, frequent occurrence of the same inhibitor often inhibiting more than one enzyme, and complex mixtures of inhibitors that affect various classes of the proteases at the same time.

In particular, the present invention relates to a method of obtaining at least one proteinase protein inhibitor from a plant material comprising the steps of extracting at least one proteinase protein inhibitor and other plant proteins from the plant material using a solvent; heating the extract to denature the plant proteins; removing the denatured plant proteins; and salting-out the remaining fraction of the plant proteinase inhibitors designated as PPI fraction from aligned fraction of the extract liquid, and desalting the PPI fraction.

It has been found that there is no need to extensively purify potato proteinase inhibitor II in order to produce a biologically active product useful in regulation of food uptake in mammals. Isolation of a crude fraction of potato proteinase inhibitors (PPI) is sufficient to achieve this goal. It is believed that part of PPI activity is attributed to yet unknown interaction between the individual components of the PPI fraction, as evident from animal feeding studies described below. By using salting out as the last step of extraction procedure, the method of the present invention recovers all heat stable potato proteins, therefore enriching the PPI fraction with various proteins in addition to proteinase inhibitor II.

Pharmaceutical compositions comprising the potato protein extract enriched with one or more protease inhibitors, as described above, and one or more pharmaceutically acceptable formulation agents are also encompassed by this invention.

In one embodiment, the present invention is directed to a therapeutic composition comprising a pharmaceutically acceptable vehicle and a potato protein extract enriched with heat stable protease inhibitors. In still another embodiment, the subject invention is directed to methods of treating or regulating appetite, food intake, and body weight in a mammalian subject comprising administering to the subject a therapeutically effective amount of the above compositions. These and other embodiments of the subject invention will be apparent to one of skill in the art in view of the disclosure herein.

### DETAILED DESCRIPTION

The present invention comprises a simplified extraction protocol for preparing biologically active heat stable potato proteinase inhibitors (PPI) from whole potato tubers. In a first step, plant proteins are extracted from plant material, such as potato tubers. In addition to potato tubers, representative plant materials can include tubers of sweet potatoes or cassava, leaves and fruits of potatoes, tomatoes and legumes, seeds of all plant species, as well as genetically engineered organisms expressing proteinase inhibitor genes, using an aqueous solvent. In one embodiment, selected tubers are added to a blender and an extractant buffer is supplied simultaneously. For example, the tubers can be added at a 40% weight basis of the extractant buffer. An example extractant buffer is sodium chloride:acetic acid:water, 1:1:9, w:v:v. The mixture is blended until liquefied, and the liquid material is recovered by centrifugation, filtering, or pressing or the like. Under these conditions most of the proteins from the PPI fraction will be extracted from the plant tissue and accumulated in the solution. In a second step, the extract liquid is heated to denature the heat sensitive plant proteins. In a third step, the extract liquid is cooled to precipitate the plant proteins. This step allows the removal of heat sensitive proteins due to coagulation. For example, the extract liquid is heated to approximately 70° C. and cooled to approximately 30° C. In a subsequent step, the liquid portion of the extract liquid is separated by centrifugation, filtering, or pressing to remove the denatured plant proteins. The volume of the liquid is recorded. Thereafter, the heat stable fraction of potato proteins is then salted out from this liquid in a subsequent step. For example, sodium chloride can be used in an amount of about 260 g/l of sodium chloride. The sample is mixed until the salt is completely dissolved. Under these conditions, the biologically active PPI fraction containing one or more protease inhibitor proteins at a variety of degrees of purity including, but not limited to, potato proteinase inhibitor II, potato proteinase inhibitor I, potato cysteine protease inhibitor, and potato carboxypeptidase inhibitor is precipitated and can be recovered by centrifugation, filtering, or pressing and is designated as “PPI fraction”. A subsequent step of the extraction protocol desalts the PPI fraction. For example, a Sephadex gel filtration column can be used. PPI fraction is loaded on the Sephadex column and eluted with about 50 mM ammonium bicarbonate in water. Finally, the PPI fraction is lyophilized to obtain a dry PPI powder, which retains its biological activity and is highly active against in controlling the appetite and food intake in mammalian subjects.

Various modes of administration, including all modes known in the art, are contemplated for use in delivering the extract enriched with one or more protease inhibitors to a mammal such as a human patient. Preferred modes of administration include parental (e.g., intravenous, intramuscular and subcutaneous), oral administration, and topical administration. The extract enriched with one or more protease inhibitors can be added to a pharmaceutical composition, nutraceutical, functional food and/or cosmetic composition in any suitable amount. In an embodiment, the pharmaceutical composition, nutraceutical, functional food and/or cosmetic composition includes the extract enriched with one or more protease inhibitors of the present invention in an amount of at least 0.5% by weight, preferably from about 1% to about 80% by weight, more preferably about 1% to about 20% by weight.

In an embodiment, the pharmaceutical compositions containing the extract of the present invention may be in any form suitable for oral use, such as e.g., tablets, troches, lozenges, aqueous or oily suspensions, dispersible powders or granules, emulsions, hard or soft capsules, or syrups or elixirs. Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient(s) in admixture with non-toxic pharmaceutically acceptable excipients, such as inert diluents, granulating, disintegrating and lubricating agents, which are suitable for the manufacture of tablets. The tablets may be uncoated or they may be coated by known techniques to delay disintegration and absorption in the gastrointestinal tract and thereby provide a sustained action over a longer period. Formulations for oral use may also be presented as hard gelatin capsules wherein the active ingredient is mixed with an inert solid diluent, or as soft gelatin capsules wherein the active ingredients is mixed with water or an oil medium. Aqueous suspensions contain the active material in admixture with excipients suitable for the manufacture of aqueous suspensions, such as e.g., suspending agents, dispersing or wetting agents, preservatives, coloring agents, flavoring agents, and sweetening agents. Dispersible powders and granules suitable for preparation of an aqueous suspension by the addition of water provide the active ingredient(s) in admixture with a dispersing or wetting agent, suspending agent and one or more preservatives. Additional excipients, for example sweetening, flavoring and coloring agents, may also be present.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

Administration of the extract enriched with one or more protease inhibitors of the present invention can also be used for treating or regulating appetite, food intake, and body weight in a mammalian subject.

Although the present invention is further described in the examples below, the scope of the present invention is not limited to that described in these examples.

### EXAMPLE 1

One kg of potato tubers was extracted with 400 ml of the extractant buffer (sodium chloride:acetic acid:water, 1:1:9, w:v:v) using a handheld commercial blender for 5 minutes. The resulting mixture was centrifuged at 13,000 g for 10 minutes and filtered through Whatman filter paper. The resulting liquid (450 ml) was transferred to an Erlenmeyer flask and heated to 70° C. on a hot plate. The solution was then cooled to approximately 30° C. and centrifuged at 13,000 g for 10 minutes to pellet the visible precipitate of unwanted denatured proteins.

The clarified potato extract (400 ml) was transferred to an Erlenmeyer flask and 104 g of sodium chloride was slowly added to the solution while stirring. The solution was kept stirring for another hour before it was centrifuged at 13,000 g for 10 minutes to pellet the visible precipitate, designated as the wet PPI fraction.

At the next step, the wet PPI fraction was dissolved in 50 ml of water and loaded onto a gel filtration column (a total bed volume of 150 ml) packed with Bio-Gel P-6 DG matrix (Bio-Rad, Hercules, Calif.). Then the PPI fraction was eluted with 50 mM ammonium bicarbonate. The first 3 fractions (20 ml each) were discarded. The next 6 fractions (20 ml each) were pooled and lyophilized. A 50 ul sample from each selected fraction was analyzed on a 15% polyacrylamide gel and proteinase inhibitory activity was visualized using a slightly modified method of Chavan and Hejgaard (1981).

The PPI fraction proteins were solubilized in water and subjected to nondenaturing gel electrophoresis to identify areas of proteinase inhibition in the gel. The gel after electrophoresis was treated with trypsin or chymotrypsin and subsequently developed in the presence of N-Acetyl-phenylalanine-naphthylester plus the dye o-Dianisidine tetrazotized for 10 minutes at 37° C.

The gel (10 cm by 8 cm) shows two major regions of inhibitory activity and several minor regions corresponding to approximately 5-40 kDa molecular weight proteins. The most prominent areas of trypsin inhibitory activity were located at the region of 20 to 25 kDa (0.64 cm²) and 40 kDa (0.41 cm²), while a weaker area of inhibitory activity associated with a 5-15 kDa region on the gel (Table 1).

TABLE 1
Results of identification of the serine protease inhibitory
activity in the potato protein extract (PPI) follwing
SDS-PAGE and activity-specific stain.

| Group | Anti-trypsin | Anti-chymotrypsin |
|---|---|---|
| (Molecular weight) | activity (cm²) | activity (cm²) |
| 40-35 kDa | 0.41 ± 0.05 | 0.35 ± 0.07 |
| 35-25 kDa | 0.11 ± 0.04 | 0.02 ± 0.01 |
| 25-15 kDa | 0.64 ± 0.09 | 0.78 ± 0.12 |
| 15-5 kDa | 0.23 ± 0.09 | 0.33 ± 0.14 |

### EXAMPLE 2

A similar result was obtained when proteins from the PPI fraction were separated on a denatured SDS-PAGE gel electrophoresis and stained with Coomassie Blue dye to reveal all proteins present in the final fraction. The proteins were solubilized in water and subjected to denaturing SDS-gel electrophoresis to identify areas of proteinase inhibition in the gel. The gel after electrophoresis was treated with trypsin and subsequently developed in the presence of N-Acetyl-phenylalanine-naphthylester plus the dye o-Dianisidine tetrazotized for 10 minutes at 37° C. The same gel was subsequently stained with Coomassie Blue dye to reveal all proteins in the sample.

Six major bands observed under these conditions were excised from the gel and subject to N-terminal sequencing for identification of the proteins present in the potato protein extract (Table 2). The complete identification of group 4 protein could not be provided due to signal interference, most probably resulting from the fact that this particular band has not been purified to homogeneity. Two proteins co-located with the areas of major serine protease inhibitory activity were successfully confirmed by MALDI-TOF analysis as potato proteinase inhibitor I and potato proteinase inhibitor II.

TABLE 2
Results of identification of the protein
present in the potato extract (PPI).

| Group | Molecular weight (kDa) | Type of protein |
|---|---|---|
| 1 | 40 | potato proteinase inhibitor II (dimer) |
| 2 | 25 | potato cysteine protease inhibitor |
| 3** | 22 | potato proteinase inhibitor II |
| 4* | 20 | potato kunitz-type protease inhibitor |
| 5** | 8 | potato proteinase inhibitor I |
| 6 | 5 | potato carboxypeptidase inhibitor |

*Protein from this group could not be positively identified due to signal interference; the most probable match is listed.
**MALDI-TOF confirmation of protein identification.

### EXAMPLE 3

In order to confirm the reduction of food intake upon the consumption of the proteins from the PPI fraction of potato, conducted a set of animal feeding studies were conducted. All studies performed so far utilized a thoroughly purified proteinase inhibitor II as a test article, largely because of the previous claims that other proteins in the test article will have no beneficial affect on the activity of that article, or will have a detrimental effect (Ausich et al., 2003). For example, U.S. Pat. No. 6,686,456 deals specifically with elimination of Kunitz family, Bowman-Birk proteinase and carboxypeptidase inhibitors from other potato proteinase inhibitors (Ausich et al., 2004). By using salting out as the last step of extraction procedure, the method of the present invention recovers all heat stable potato proteins, therefore enriching the PPI fraction with various proteins in addition to proteinase inhibitor II.

In Study 1, Male Wistar rats (n=24, age=8 weeks, mean body weight=250 g) were obtained from Charles River Laboratories (St. Constant, Canada). The rats were housed individually in the wire cages and were maintained in an air-conditioned environment at 23±2° C. with a 12 h light-dark cycle (lights on at 2 am). The rats were fed AIN-93 standard diet and tap water ad libidum, and were adapted to 6 h of food deprivation that began at 8 am. All animals were weighed daily, and food consumed by each animal was determined by differential weighing. On days 3-6 after arrival all animals were adapted to gavage with water. Experiments began on day 7 when it was determined that the process of gavaging did not significantly affect the daily food uptake.

Animals were randomly allocated in 2 groups. Each group received either a control treatment (2 ml of water) or 100 mg/kg dose of PPI fraction dissolved in 2 ml of water. All treatments were given prior to access to food at 2 pm, which is also the beginning of the night cycle. Food consumption was measured to the nearest 0.1 g, at 1 h, 2 h, and 24 h (daily intake) after the treatment (Table 3).

TABLE 3
Single-dose oral PPI treatment (100 mg/kg) significantly
reduced both immediate (1 h and 2 h since ingestion) and
daily (24 h) food intake in male rats (p < 0.05).
Data is expressed as a % of reduction of food intake in
treated animals as compared to the control group.

| Time past treatment (hours) | % Reduction of Food Intake |
|---|---|
| 1 | 40% ± 8% |
| 2 | 42% ± 9% |
| 24 | 11% ± 4% |

Higher levels of circulating CCK, induced by the proteins from PPI fraction, are a likely cause for the reduced food consumption. It is well established that CCK levels gradually increase 20-30 minutes after meal initiation and then gradually fall for the next 2 to 3 hours. The animals in the treatment group showed reduced food intake, by almost 40%, during the first 2 hours after they were presented with a meal. This observation is strongly suggestive of a satiety effect mediated by CCK.

### EXAMPLE 4

In Study 2, animals were randomly allocated in 2 groups. Each group received either a control treatment (2 ml of water) or 100 mg/kg dose of PPI fraction dissolved in 2 ml of water for 10 consecutive days. All treatments were given prior to access to food at 2 pm, which is also the beginning of the night cycle. Food consumption and body weight were recorded daily prior to the 2 pm treatment (Table 4).

TABLE 4
Repeated PPI administration (100 mg/kg for 10 days) significantly
reduced cumulative food intake during the study period. A significant
reduction in animal body weight gain as compared to the control group
was also observed. Data is expressed as a % of reduction of food intake
in treated animals as compared to the control group.

| Treatment | % Reduction of Food | |
|---|---|---|
| Group | Intake | % Reduction of Weight Gain |
| Control | 0% ± 2% | 0% ± 4% |
| 100 mg/kg/day | 6% ± 2% | 11% ± 5% |

Previously (Peikin, 1986) it was found that the administration of 100 mg/kg of purified trypsin inhibitor for 7 days decreased daily food intake in rats on average by 2.4% (obese animals) and by 2% (lean animals). The above data demonstrates that when applied orally, proteins from the PPI fraction reduce the animal body weight gain by up to 11% during the 10-day study. The reduction can be partially explained by a 6% decrease in energy (food) intake, indicating that PPI, a complex mixture of thermostable inhibitors from potato, is more effective in regulating food intake and body weight than the purified trypsin inhibitor alone. Although our finding build on the previous studies in different animal models that show that orally administered proteinase inhibitors interfere with a typical satiation response, they also suggest that there exists yet unknown interaction and/or activity within the proteins of PPI fraction that is responsible for additional reduction in weight gain.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A method for obtaining at least one heat stable plant proteinase inhibitor from plant material comprising the steps of:
(a) extracting the at least one heat stable plant proteinase inhibitor and other plant proteins from the plant material using an extraction solvent;
(b) heating the extract to denature at least some of the plant proteins;
(c) removing the denatured plant proteins from the extract; and
(d) salting-out the at least one heat stable plant proteinase inhibitors designated as PPI fraction from a liquid fraction of the extract.

2. The method of claim 1 wherein the extraction solvent is an aqueous solvent.

3. The method of claim 2 wherein the extraction solvent comprises acetic acid and sodium chloride.

4. The method of claim 1 wherein in step (c) said denatured plant proteins are removed by the steps of:
cooling the extract to precipitate the denatured plant proteins; and
performing a separation process on the cooled extract.

5. The method of claim 4 wherein the separation process on the cooled extract is performed by one or more steps of centrifuging the cooled extract, filtering the cooled extract, and pressing the cooled extract.

6. The method of claim 1 after step (d) further comprising the step of:
desalting the PPI fraction.

7. The method of claim 6 wherein said step of desalting the PPI fraction is performed by gel filtration.

8. The method of claim 7 wherein a buffer used in said step of desalting the PPI fraction comprises ammonium bicarbonate.

9. The method of claim 7 wherein said step of desalting the PPI fraction is performed by freeze-drying.

10. The method of claim 1 wherein the plant material is selected from the members of the Solanaceae family.

11. The method of claim 1 wherein the plant material is potato tubers.

12. The method of claim 1 wherein said at least one heat stable protease inhibitor is selected from the group consisting of potato proteinase inhibitor II, potato proteinase inhibitor I, potato cysteine protease inhibitor, and potato carboxypeptidase inhibitor.

13. A therapeutic composition comprising a pharmaceutically acceptable vehicle and a potato protein extract enriched in said at least one heat stable protease inhibitor obtained by the method of claim 1.

14. The therapeutic composition of claim 13 wherein the at least one heat stable protease inhibitor is selected from the group consisting of potato proteinase inhibitor II, potato proteinase inhibitor I, potato cysteine protease inhibitor, and potato carboxypeptidase inhibitor.

15. A method of controlling or regulating one or more appetite, food intake or body weight in a mammalian subject comprising the steps of:
administering to said subject a therapeutically effective amount of a composition of at least one protease inhibitor wherein the at least one protease inhibitor protein is obtained by a method comprising the steps of:
(a) extracting the at least one heat stable plant proteinase inhibitor and other plant proteins from the plant material using an extraction solvent;
(b) heating the extract to denature at least some of the plant proteins;
(c) removing the denatured plant proteins from the extract; and
(d) salting-out the at least one heat stable plant proteinase inhibitors designated as PPI fraction from a liquid fraction of the extract.

16. The method of claim 15 wherein the extraction solvent is an aqueous solvent.

17. The method of claim 16 wherein the extraction solvent comprises acetic acid and sodium chloride.

18. The method of claim 17 wherein in step (c) said denatured plant proteins are removed by the steps of:
cooling the extract to precipitate the denatured plant proteins; and
performing a separation process on the cooled extract.

19. The method of claim 17 wherein the separation process on the cooled extract is performed by one or more steps of centrifuging the cooled extract, filtering the cooled extract, and pressing the cooled extract.

20. The method of claim 15 wherein after step (d) further comprising the step of:
desalting the PPI fraction.

21. The method of claim 20 wherein said step of desalting the PPI fraction is performed by gel filtration.

22. The method of claim 21 wherein a buffer used in said step of desalting the PPI fraction comprises ammonium bicarbonate.

23. The method of claim 20 wherein said step of desalting the PPI fraction is performed by freeze-drying.

24. The method of claim 15 wherein the plant material is selected from the members of the Solanaceae family.

25. The method of claim 15 wherein the plant material is potato tubers.

26. The method of claim 15 wherein said at least one heat stable protease inhibitor is selected from the group consisting of potato proteinase inhibitor II, potato proteinase inhibitor I, potato cysteine protease inhibitor, and potato carboxypeptidase inhibitor.

27. The method of claim 15 wherein the step of administering to said subject is performed orally.

28. The method of claim 15 wherein the mammal is a human.

29. A pharmaceutical product for controlling or regulating body weight comprising a composition of at least one protease inhibitor wherein the at least one protease inhibitor is obtained by a method comprising the steps of:
(a) extracting the at least one heat stable plant proteinase inhibitor and other plant proteins from the plant material using an extraction solvent;
(b) heating the extract to denature at least some of the plant proteins;
(c) removing the denatured plant proteins from the extract; and
(d) salting-out the at least one heat stable plant proteinase inhibitors designated as PPI fraction from a liquid fraction of the extract.

30. The pharmaceutical product of claim 29 wherein the extraction solvent is an aqueous solvent.

31. The pharmaceutical product of claim 30 wherein the extraction solvent comprises acetic acid and sodium chloride.

32. The pharmaceutical product of claim 29 wherein in step (c) said denatured plant proteins are removed by the steps of:
cooling the extract to precipitate the denatured plant proteins; and
performing a separation process on the cooled extract.

33. The pharmaceutical product of claim 32 wherein the separation process on the cooled extract is performed by one or more steps of centrifuging the cooled extract, filtering the cooled extract, and pressing the cooled extract.

34. The pharmaceutical product of claim 29 wherein after step (d) further comprising the step of:
desalting the PPI fraction.

35. The pharmaceutical product of claim 34 wherein said step of desalting the PPI fraction is performed by gel filtration.

36. The method of claim 35 wherein a buffer used in said step of desalting the PPI fraction comprises ammonium bicarbonate.

37. The pharmaceutical product of claim 34 wherein said step of desalting the PPI fraction is performed by freeze-drying.

38. The pharmaceutical product of claim 29 wherein the plant material is selected from the members of the Solanaceae family.

39. The pharmaceutical product of claim 29 wherein the plant material is potato tubers.

40. The pharmaceutical product of claim 29 wherein said at least one heat stable protease inhibitors is selected from the group consisting of potato proteinase inhibitor II, potato proteinase inhibitor I, potato cysteine protease inhibitor, and potato carboxypeptidase inhibitor.

41. A method for obtaining at least one proteinase inhibitor from plant material comprising the steps of:
(a) extracting plant proteins from the plant material using an aqueous extraction solvent;
(b) heating the extract to denature the plant proteins;
(c) cooling the extract to precipitate the denatured plant proteins;
(d) performing a separation process on the cooled extract to remove the denatured plant proteins from a liquid fraction of the extract;
(e) salting-out the remaining fraction of the plant proteinase inhibitors designated as PPI fraction from the liquid fraction; and
(f) desalting the PPI fraction.
